(19) United States
(12) Patent Application Publication
(10) Pub. No.: US 2026/0321417 A1
(43) Pub. Date: Oct. 15, 2026

(54) METHODS FOR PREDICTING CANCER-ASSOCIATED VENOUS THROMBOEMBOLISM ACROSS MULTIPLE CANCER TYPES

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Simon Mantha, New York, NY (US); Subrata Chatterjee, New York, NY (US); Rohan Singh, New York, NY (US); John Cadley, New York, NY (US)

(21) Appl. No.: 19/104,209

(22) PCT Filed: Aug. 16, 2023
(86) PCT No.: PCT/US2023/072330

§ 371 Date: Feb. 15, 2025

Related U.S. Application Data
(60) Provisional application No. 63/398,628, filed on Aug. 17, 2022.

(51) Int. Cl. G16H 50/20; G16B 40/30

(52) U.S. Cl. CPC G16H 50/20; G16B 40/30

(57) ABSTRACT

The present disclosure relates generally to methods, devices, and systems for accurately estimating the risk of cancer-associated venous thromboembolism across multiple cancer types.

### CROSS-REFERENCE TO RELATED APPLICATIONS

This application claims the benefit of and priority to U.S. Provisional Patent Application No. 63/398,628 filed Aug. 17, 2022, the entire contents of which are incorporated herein by reference.

### STATEMENT OF GOVERNMENT SUPPORT

This invention was made with government support under grant number CA008748 awarded by National Institutes of Health. The government has certain rights in the invention.

### TECHNICAL FIELD

The present technology relates generally to methods, devices, and systems for accurately estimating the risk of cancer-associated venous thromboembolism across multiple cancer types.

### BACKGROUND

The following description of the background of the present technology is provided simply as an aid in understanding the present technology and is not admitted to describe or constitute prior art to the present technology.

Cancer has long been known to confer an increased risk of venous thromboembolism (VTE).¹ The pathophysiological mechanisms are complex and remain incompletely elucidated.² Cancer-associated VTE is common, as approximately 20% to 30% of VTE episodes are associated with a malignancy.³ Those events are clinically important, as they are a leading cause of mortality in patients with cancer.⁴ Several randomized trials have demonstrated the effectiveness of pharmacological prophylaxis, however applicability has been limited by currently available VTE risk stratification tools.^{5,6} Recent evidence suggests that tumor somatic genetic alterations influence the risk of VTE.²⁰⁻⁴² Notably, in some cases, genes specific effects appear to be conditional to tumor type. Additionally, available data suggests an interaction of multiple genes, each contributing a small amount of information to risk prediction rather than a single gene mediating a large part of the risk—this highlights the complex interactions between these genomic alterations and the need to integrate data on multiple covariates.

Accordingly, there is an urgent need for accurate methods for accurately estimating the risk of cancer-associated venous thromboembolism across multiple cancer types.

### SUMMARY OF THE PRESENT TECHNOLOGY

In one aspect, the present disclosure provides a method of training a machine learning classifier for estimating risk of cancer-associated venous thromboembolism (VTE) in cancer patients comprising: (a) receiving data on a cohort of subjects, the subjects in the cohort having a plurality of cancer types; (b) generating a training dataset based on the received data, wherein the training dataset comprises a plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and (c) applying a machine learning method to the training dataset to develop the machine learning classifier for estimating risk of cancer-associated VTE in cancer patients, wherein applying the machine learning method comprises: applying a machine learning technique to the training dataset; performing hyperparameter optimization to identify one or more machine learning models with an accuracy that exceeds an accuracy threshold for the classifier; and determining an optimal operating-point threshold based on optimization of sensitivity and specificity of the receiver operating characteristic (ROC) curves for the training dataset; wherein the classifier is configured to receive the plurality of features for cancer patients and generate predictors for risk of cancer-associated VTE in cancer patients. The subjects in the cohort may be chemotherapy-naïve or may have received systemic chemotherapy. Additionally or alternatively, in certain embodiments, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma. In any and all embodiments of the methods disclosed herein, the plurality of features does not comprise activated partial thromboplastin time, prothrombin time, or body mass index (BMI).

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

Additionally or alternatively, in some embodiments of the methods disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

In any of the preceding embodiments, the method further comprises applying the classifier to data on a cancer patient to generate a predictor, and determining whether the cancer patient is at risk for cancer-associated VTE based on the predictor and the operating-point threshold. In some embodiments, the predictor comprises a cumulative incidence function (CIF) for cancer-associated VTE.

In any of the foregoing embodiments, the method further comprises administering an effective amount of anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in some embodiments of the methods disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In one aspect, the present disclosure provides a method of estimating risk of cancer-associated venous thromboembolism (VTE) in a cancer patient using a machine learning classifier, the method comprising: receiving patient data corresponding to a plurality of features for the cancer patient; applying the machine learning classifier to the patient data to generate a predictor; and determining whether the cancer patient is at risk for cancer-associated VTE based on the predictor and an operating-point threshold, wherein the machine learning classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. In some embodiments, the method further comprises administering an effective amount of anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, and enoxaparin. Additionally or alternatively, in some embodiments, the predictor comprises a cumulative incidence function (CIF) for cancer-associated VTE. The subjects in the cohort may be chemotherapy-naïve or may have received systemic chemotherapy. In any of the preceding embodiments of the methods disclosed herein, the plurality of features for the cancer patient are determined by assaying blood and/or sequencing tumor DNA.

Additionally or alternatively, in certain embodiments, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma. In any and all embodiments of the methods disclosed herein, the plurality of features does not comprise activated partial thromboplastin time, prothrombin time, or body mass index (BMI).

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

Additionally or alternatively, in some embodiments of the methods disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

Additionally or alternatively, in some embodiments of the methods disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In any and all embodiments of the methods disclosed herein, the plurality of features for each subject in the cohort are determined by assaying blood and/or sequencing tumor DNA.

In any and all embodiments of the methods disclosed herein, the cancer-associated VTE is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

In another aspect, the present disclosure provides a machine learning system for training a machine learning classifier for estimating risk of cancer-associated venous thromboembolism (VTE) in cancer patients, the system comprising a processor and a memory with instructions which, when executed by the processor, cause the processor to: (a) receive data on a cohort of subjects, the subjects in the cohort having a plurality of cancer types; (b) generate a training dataset based on the received data, wherein the training dataset comprises a plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent, and wherein the plurality of features does not include neutrophil count and platelet count; and (c) apply a machine learning method to the training dataset to develop the machine learning classifier for estimating risk of cancer-associated VTE in cancer patients; wherein applying the machine learning method comprises: applying a machine learning technique to the training dataset; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. The subjects in the cohort may be chemotherapy-naïve or may have received systemic chemotherapy.

In any and all embodiments of the systems disclosed herein, the plurality of features does not comprise activated partial thromboplastin time, prothrombin time, or body mass index (BMI).

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

Additionally or alternatively, in some embodiments of the systems disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

Additionally or alternatively, in certain embodiments of the systems disclosed herein, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

In any of the preceding embodiments of the systems described herein, the instructions further cause the processor to apply the machine learning classifier to data on a cancer patient to generate a predictor, and determine whether the cancer patient is at risk for cancer-associated VTE based on the predictor and the operating-point threshold. In some embodiments, the predictor comprises a cumulative incidence function (CIF) for cancer-associated VTE.

In any of the foregoing embodiments of the systems described herein, the instructions further cause the processor to recommend an anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in some embodiments of the systems disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In yet another aspect, the present disclosure provides a computing system for estimating risk of cancer-associated venous thromboembolism (VTE) in a cancer patient, the computing system comprising a processor and a memory with instructions which, when executed by the processor, cause the processor to: receive patient data corresponding to a plurality of features for the cancer patient; apply a machine learning classifier to the patient data to generate a predictor; and determine whether the cancer patient is at risk for cancer-associated VTE based on the predictor and an operating-point threshold, wherein the classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

In any and all embodiments of the systems disclosed herein, the plurality of features does not comprise activated partial thromboplastin time, prothrombin time, or body mass index (BMI).

Additionally or alternatively, in some embodiments of the systems disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

In any of the preceding embodiments of the systems described herein, the instructions further cause the processor to recommend an anticoagulant therapy to the cancer patient predicted to be at risk for cancer-associated VTE based on the predictor and the operating-point threshold. In some embodiments, the predictor comprises a cumulative incidence function (CIF) for cancer-associated VTE. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in certain embodiments of the systems disclosed herein, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

Additionally or alternatively, in some embodiments of the systems disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In any and all embodiments of the systems disclosed herein, the plurality of features for each subject in the cohort are determined by assaying blood and/or sequencing tumor DNA.

In one aspect, the present disclosure provides a non-transitory computer-readable storage medium comprising instructions which, when executed by a processor of a machine learning system, configure the machine learning system to train a machine learning classifier to estimate risk of cancer-associated venous thromboembolism (VTE) in cancer patients, wherein the instructions are configured to cause the processor to: (a) receive data on a cohort of subjects, the subjects in the cohort having a plurality of cancer types; (b) generate a training dataset based on the received data, wherein the training dataset comprises a plurality of features for each subject in the cohort, the plurality of features comprising (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent, and wherein the plurality of features does not include neutrophil count and platelet count; and (c) apply a machine learning method to the training dataset to develop the machine learning classifier for estimating risk of cancer-associated VTE in cancer patients; wherein applying the machine learning method comprises: applying a machine learning technique to the training dataset; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. The subjects in the cohort may be chemotherapy-naïve or may have received systemic chemotherapy.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

In any and all embodiments of the computer-readable storage medium disclosed herein, the plurality of features does not comprise activated partial thromboplastin time, prothrombin time, or body mass index (BMI).

Additionally or alternatively, in some embodiments of the computer-readable storage medium disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

In any of the preceding embodiments of the computer-readable storage medium described herein, the instructions further cause the processor to apply the machine learning classifier to data on a cancer patient to generate a predictor, and determine whether the cancer patient is at risk for cancer-associated VTE based on the predictor and the operating-point threshold. In some embodiments, the predictor comprises a cumulative incidence function (CIF) for cancer-associated VTE.

Additionally or alternatively, in certain embodiments of the computer-readable storage medium disclosed herein, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

In any of the preceding embodiments of the computer-readable storage medium described herein, the instructions further cause the processor to recommend an anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in some embodiments of the computer-readable storage medium disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In another aspect, the present disclosure provides a non-transitory computer-readable storage medium comprising instructions which, when executed by a processor of a computing system, configure the computing system to estimate risk of cancer-associated venous thromboembolism (VTE) in a cancer patient, wherein the instructions are configured to cause the processor to: receive patient data corresponding to a plurality of features for the cancer patient; apply a machine learning classifier to the patient data to generate a predictor; and determine whether the cancer patient is at risk for cancer-associated VTE based on the predictor and an operating-point threshold, wherein the classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

In any and all embodiments of the computer-readable storage medium disclosed herein, the plurality of features does not comprise activated partial thromboplastin time, prothrombin time, or body mass index (BMI).

Additionally or alternatively, in some embodiments of the computer-readable storage medium disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

In any of the preceding embodiments of the computer-readable storage medium described herein, the instructions further cause the processor to recommend an anticoagulant therapy to the cancer patient predicted to be at risk for cancer-associated VTE based on the predictor and the operating-point threshold. In some embodiments, the predictor comprises a cumulative incidence function (CIF) for cancer-associated VTE. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in certain embodiments of the computer-readable storage medium disclosed herein, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

Additionally or alternatively, in some embodiments of the computer-readable storage medium disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In any of the preceding embodiments of the computer-readable storage medium disclosed herein, the plurality of features for the cancer patient are determined by assaying blood and/or sequencing tumor DNA.

### BRIEF DESCRIPTION OF THE DRAWINGS

FIG. 1 shows a flow diagram of cohort selection. * refers to a first sub-cohort consisting of adults with blood control drawn for MSK-IMPACT™ analysis between 2014 and 2016. † refers to a second sub-cohort consisting of adults with blood control drawn for MSK-IMPACT™ analysis between 2017 and 2019. ‡ refers to patients randomly allocated between training and validation sets, stratified by event type.

FIG. 2 shows distribution of times from cancer diagnosis to cohort entry. Cancer diagnosis time corresponds to first pathological evidence of neoplasia and cohort entry is defined by report of MSK-IMPACT™ results.

FIG. 3 shows cancer-associated VTE cumulative incidence functions. Cumulative incidence functions were derived from the Kaplan-Meier (KM) and the competing risk (CR) estimators.

FIG. 4 shows an exemplary receiver operating characteristic (ROC) curve. ROC plot was computed using the DeepHit model featuring a “limited” set of covariates (which include the BASIC, CHEMO, BASIC_LAB, and ADDITONAL_LAB elementary sets; see Example 1) fitted on the training set and evaluated on the validation set.

FIG. 5A shows an exemplary calibration curve that was computed using the DeepHit model featuring a “limited” set of covariates (which include the BASIC, CHEMO, BASIC_LAB, and ADDITONAL_LAB elementary sets; see Example 1) fitted on the training set and evaluated on the validation set.

FIG. 5B shows an exemplary calibration curve that was computed using the DeepHit model featuring an “extensive” set of covariates (which include the BASIC, CHEMO, GENETIC, BASIC_LAB, and ADDITONAL_LAB elementary sets; see Example 1) fitted on the training set and evaluated on the validation set.

FIG. 6A is a block diagram depicting an embodiment of a network environment comprising a client device in communication with server device.

FIG. 6B is a block diagram depicting a cloud computing environment comprising client device in communication with cloud service providers.

FIGS. 6C and 6D are block diagrams depicting embodiments of computing devices useful in connection with the methods and systems described herein.

FIG. 7 depicts a system that includes a computing device and a sample processing system according to various potential embodiments.

FIG. 8 shows the results for VTE on the Training Set for each of the tested 11 covariate sets.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

VTE is an important complication of cancer for which effective pharmacological prophylaxis methods exist. However, currently available prediction rules have limited accuracy in stratifying patients for VTE risk. Accordingly, approaches to enhance the overall benefit of VTE prophylaxis in cancer patients will be contingent on improved methods to quantify risk.

The present disclosure demonstrates that the machine learning methods, devices, and systems described herein are capable of accurately estimating the risk of cancer-associated VTE across multiple cancer types, and informing future therapeutic interventions. These results were unexpected because the machine learning methods of the present technology actually exclude features such as leukocyte counts (e.g., neutrophil count), and platelet count, which serve as core predictors of conventional algorithms for predicting VTE risk (e.g., Khorana Score (KS)).

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value)

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. In some embodiments, the cancer is bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, or soft tissue sarcoma.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

“Negative predictive value (NPV)” is defined as the proportion of subjects with a negative test result who are correctly identified. A high NPV means that when the test yields a negative result, it is unlikely that the result should have been positive. The NPV is determined as:

$$NPV = \frac{\# of True Negatives}{( \# of True Negatives + \# of False Negatives )} = \frac{\# of True Negatives}{\# of Negatives calls}$$

where a “true negative” is the event that the test makes a negative prediction, and the subject has a negative result under the gold standard, and a “false negative” is the event that the test makes a negative prediction, and the subject has a positive result under the gold standard.

The “positive predictive value (PPV),” or “precision rate” is a summary statistic used to describe the proportion of subjects with positive results who are correctly identified. It is a measure of the performance of a predictive method, as it reflects the probability that a positive result reflects the underlying condition being tested for. Its value does however depend on the prevalence of the outcome of interest, which may be unknown for a particular target population. The PPV can be derived using Bayes' theorem. The PPV is defined as:

$$PPV = \frac{\# of True Positives}{( \# of True Positives + \# of False Positives )} = \frac{\# of True Positives}{\# of Positives calls}$$

where a “true positive” is the event that the predictive test makes a positive prediction, and the subject has a positive result under the gold standard, and a “false positive” is the event that the test makes a positive prediction, and the subject has a negative result under the gold standard.

If the prevalence, sensitivity, and specificity are known, the positive and negative predictive values (PPV and NPV) can be calculated for any prevalence as follows:

$$PPV = \frac{sensitivity \times prevalence}{sensitivity \times prevalence + ( 1 - specificity ) \times ( 1 - prevalence )}$$

$$NPV = \frac{specificity \times ( 1 - prevalence )}{( 1 - sensitivity ) \times prevalence + specificity \times ( 1 - prevalence )}$$

If the prevalence of the disease is very low, the positive predictive value will not be close to 1, even if both the sensitivity and specificity are high. Thus in screening the general population it is inevitable that many people with positive test results will be false positives. The rarer the abnormality, the higher the certainty that a negative test indicates no abnormality, and the lower the certainty that a positive result truly indicates an abnormality. The prevalence can be interpreted as the probability before the test is carried out that the subject has the disease, known as the prior probability of disease. The positive and negative predictive values are the revised estimates of the same probability for those subjects who are positive and negative on the test, and are known as posterior probabilities. The difference between the prior and posterior probabilities is one way of assessing the usefulness of the test.

For any test result, one can compare the probability of obtaining that result if the patient truly had the condition of interest with the corresponding probability if he or she were healthy. The ratio of these probabilities is called the likelihood ratio, calculated as sensitivity/(1−specificity). (Altman D G, Bland J M (1994). BMJ 309 (6947):102).

In statistics, sensitivity and specificity are statistical measures of the performance of a binary classification test. “Sensitivity” (also called “recall rate”) measures the proportion of actual positives which are correctly identified as such (e.g. the percentage of subjects who are correctly identified as having a condition). Sensitivity relates to the ability of a predictive test to identify positive results and is computed as the number of true positives divided by the sum of the number of true positives and the number of false negatives. “Specificity” measures the proportion of negatives which are correctly identified (e.g., the percentage of subjects who are correctly identified as not having the condition). Specificity relates to the ability of a predictive test to identify negative results and is computed as the number of true negatives divided by the sum of the number of true negatives and the number of false positives. Sensitivity and specificity are closely related to the concepts of type I and type II errors. A theoretical, optimal prediction aims to achieve 100% sensitivity and 100% specificity, however theoretically any predictor will possess a minimum error bound known as the Bayes error rate.

For any test, there is usually a trade-off between sensitivity and specificity, which can be represented graphically using a receiver operating characteristic (ROC) curve. In some embodiments, a ROC is used to generate a summary statistic. Some common versions are: the intercept of the ROC curve with the line at 90 degrees to the no-discrimination line (also called Youden's J statistic); the area between the ROC curve and the no-discrimination line; the area under the ROC curve, or “AUC” (“Area Under Curve”), or A′ (pronounced “a-prime”); d′ (pronounced “d-prime”), the distance between the mean of the distribution of activity in the system under noise-alone conditions and its distribution under signal-alone conditions, divided by their standard deviation, under the assumption that both these distributions are normal with the same standard deviation. Under these assumptions, it can be proved that the shape of the ROC depends only on d′.

As used herein, the term “overall survival” or “OS” means the observed length of life from the start of treatment to death or the date of last contact.

As used herein, “progression free survival” or “PFS” is the time from treatment to the date of the first confirmed disease progression per RECIST 1.1 and immune-related RECIST (irRECIST) criteria.

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).”

The irRECIST overall tumor assessment is based on total measurable tumor burden (TMTB) of measured target and new lesions, non-target lesion assessment and new non-measurable lesions. At baseline, the sum of the longest diameters (SumD) of all target lesions (up to 2 lesions per organ, up to total 5 lesions) is measured. At each subsequent tumor assessment (TA), the SumD of the target lesions and of new, measurable lesions (up to 2 new lesions per organ, total 5 new lesions) are added together to provide the TMTB

Overall Assessments by irRECIST

| | |
|---|---|
| Complete Response (irCR) | Complete disappearance of all measurable and non- |
| | measurable lesions. Lymph nodes must decrease to <10 |
| | mm in short axis. |
| Partial Response (irPR) | Decrease of ≥30% in TMTB relative to baseline, non-target |
| | lesions are irNN, and no unequivocal progression of new |
| | nonmeasurable lesions |
| | If new measurable lesions appear in subjects with no |
| | target lesions at baseline, irPD will be assessed. That irPD |
| | time point will be considered a new baseline, and all |
| | subsequent time points will be compared to it for response |
| | assessment. irPR is possible if the TMTB of new |
| | measurable lesions decreases by ≥30% compared to the |
| | first irPD documentation |
| | irRECIST can be used in the adjuvant setting, in |
| | subjects with no visible disease on CT/MRI scans. The |
| | appearance of new measurable lesion(s) automatically leads |
| | to an increase in TMTB by 100% and leads to irPD. These |
| | subjects can achieve a response if the TMTB decreases at |
| | follow-up, as a sign of delayed response. |
| | Based on the above, sponsors may consider |
| | enrolling subjects with no measurable disease and/or no |
| | visible disease in studies with response related endpoints. |
| Stable Disease (irSD) | Failure to meet criteria for irCR or irPR in the absence of |
| | irPD |
| Progressive Disease (irPD) | Minimum 20% increase and minimum 5 mm absolute |
| | increase in TMTB compared to nadir, or irPD for non-target |
| | or new non-measurable lesions. Confirmation of |
| | progression is recommended minimum 4 weeks after the |
| | first irPD assessment. An irPD confirmation scan may be |
| | recommended for subjects with a minimal TMTB %- |
| | increase over 20% and especially during the flare time- |
| | window of the first 12 weeks of treatment, depending on the |
| | compound efficacy expectations, to account for expected |
| | delayed response. |
| | In irRECIST a substantial and unequivocal increase |
| | of non-target lesions is indicative of progression. |
| | IrPD may be assigned for a subject with multiple |
| | new non-measurable lesions if they are considered to be a |
| | sign of unequivocal massive worsening |
| Other | irNE: used in exceptional cases where insufficient data |
| | exist, |
| | irND: in adjuvant setting when no disease is detected |
| | irNN:, no target disease was identified at baseline, and at |
| | follow-up the subject fails to meet criteria for irCR or irPD |

As used herein, the terms “subject”, “patient”, or “individual” can be an individual organism, a vertebrate, a mammal, or a human. In some embodiments, the subject, patient or individual is a human.

As used herein, “survival” refers to the subject remaining alive, and includes overall survival as well as progression free survival.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

As used herein, the terms “tumor mutation burden” or “TMB” refer to the level, e.g., number, of an alteration (e.g., one or more alterations, e.g., one or more somatic alterations) per a preselected unit (e.g., per megabase) in a predetermined set of genes (e.g., in the coding regions of the predetermined set of genes) in a tumor. Tumor mutation burden can be measured, e.g., on a whole genome or exome basis, or on the basis of a subset of genome or exome. In certain embodiments, the tumor mutation burden measured on the basis of a subset of genome or exome can be extrapolated to determine a whole genome or exome mutation burden.

In certain embodiments, the tumor mutation burden is measured in a tumor sample (e.g., a tumor sample or a sample derived from a tumor), from a subject. In certain embodiments, the tumor mutation burden is expressed as a percentile, e.g., among the mutation burden in samples from a reference population. In certain embodiments, the reference population includes patients having the same type of cancer as the subject. In other embodiments, the reference population includes patients who are receiving, or have received, the same type of therapy, as the subject. In certain embodiments, the TMB correlates with the whole genome or exome mutation load.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Conventional Algorithms for Predicting VTE Risk in Cancer Patients

The most commonly used approach to estimate the risk of cancer-associated VTE is the Khorana Score (KS), a clinical prediction rule based on cancer type, peripheral blood cell counts (i.e., platelet, erythrocyte (red blood cells), and leukocyte counts (white blood cells)), and body mass index.⁷ The KS was originally derived from a cohort of patients who had not yet received chemotherapy and were planned to start such treatments in the near future. Using this prediction rule, patients are assigned to one of three categories denoting their risk of VTE at 6 months. The KS has been extensively validated in multiple different healthcare systems.⁸ In one large review, for a KS greater or equal to 2 and 3, sensitivity was 55.2% and 23.4% respectively, while positive predictive value (PPV) was 8.9% and 11.0% respectively.⁸ However, KS is strongly dependent on tumor type and does not consider treatment-related factors influencing VTE development.

Several other clinical prediction rules have been derived by different groups⁹⁻¹⁴, which are based on the predictors already included in the KS, in addition to other clinical or tumor-specific characteristics, routine laboratory test results, presence of germline thrombophilia mutations and chemotherapy administered. In all cases those algorithms have been derived in chemotherapy-naïve patients at the time systemic therapy was planned to start. However, these clinical prediction rules are simplistic because they are limited by reliance on human computation¹⁶ and thus cannot automatically identify complex interactions between predictors which are difficult to elucidate by traditional statistical methods under human supervision.

### Systems, Devices, and Methods for Predicting the Risk of VTE Across Multiple Cancer Types

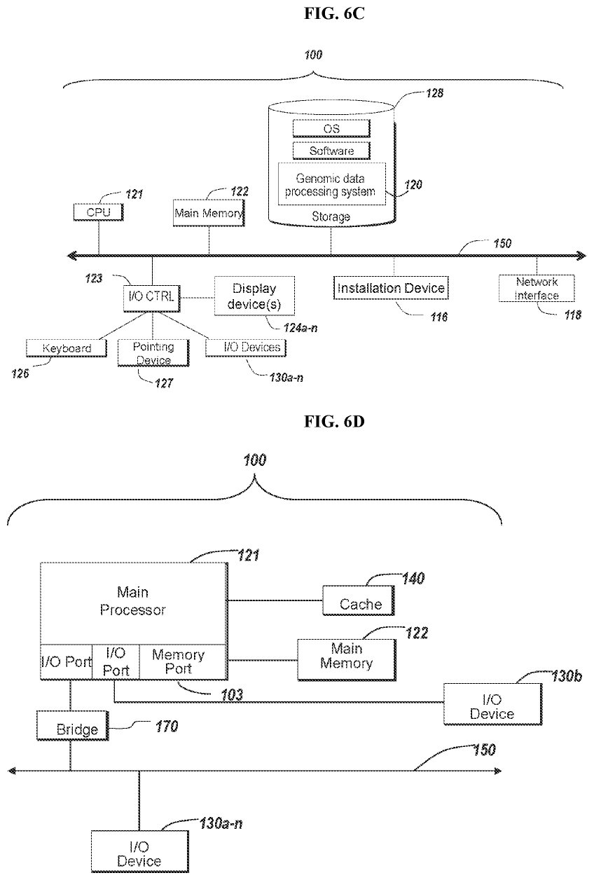

Aspects of the operating environment as well as associated system components (e.g., hardware elements) in connection with various embodiments of the methods and systems described herein will now be discussed. Referring to FIG. 6A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 6A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, 4G, or 5G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 6B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 6C and 6D depict block diagrams of a computing device 100 useful for practicing an embodiment of the client 102 or a server 106. As shown in FIGS. 6C and 6D, each computing device 100 includes a central processing unit 121, and a main memory unit 122. As shown in FIG. 6C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a genomic data processing system 120. As shown in FIG. 6D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit or memory device 122 may include one or more memory chips capable of storing data and allowing any storage location to be directly accessed by the microprocessor 121. Main memory unit or device 122 may be volatile and faster than storage 128 memory. Main memory units or devices 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 6C, the processor 121 communicates with main memory 122 via a system bus 150 (described in more detail below). FIG. 6D depicts an embodiment of a computing device 100 in which the processor communicates directly with main memory 122 via a memory port 103. For example, in FIG. 6D the main memory 122 may be DRDRAM.

FIG. 6D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 6D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 6D depicts an embodiment of a computer 100 in which the main processor 121 communicates directly with I/O device 130b or other processors 121′ via HYPERTRANSPORT, RAPIDIO, or INFINIBAND communications technology. FIG. 6D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 6C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 6C, the computing device 100 may comprise a storage device 128 (e.g. one or more hard disk drives or redundant arrays of independent disks) for storing an operating system or other related software, and for storing application software programs such as any program related to the software for the genomic data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 6B and 6C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, WINDOWS 8, and WINDOWS 10, all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

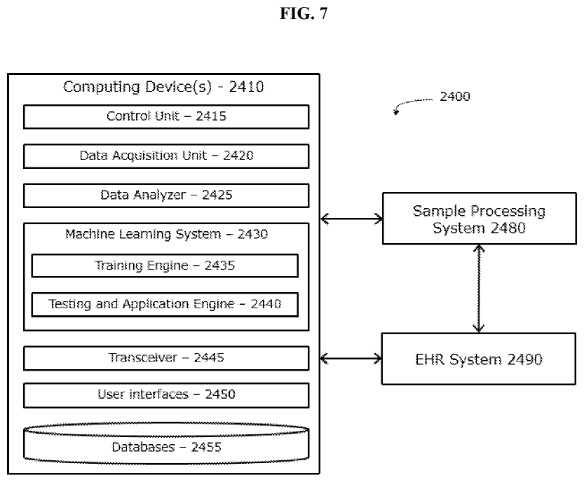

Referring to FIG. 7, in various embodiments, a system 2400 may include a computing device 2410 (or multiple computing devices, co-located or remote to each other), a sample processing system 2480, and an electronic health record (EHR) system 2490. In various embodiments, computing device 2410 (or components thereof) may be integrated with the sample processing system 2480 (or components thereof) and/or EHR system 2490 (or components thereof). In various embodiments, the sample processing system 2480 may include, may be, or may employ, in situ hybridization, PCR, Next-generation sequencing, Northern blotting, microarray, dot or slot blots, FISH, Western blotting, ELISA, colorimetric dye binding assays, complete blood count (CBC) panels, electrophoresis, chromatography, and/or mass spectroscopy on such biological sample as blood, plasma, serum, and/or tissue and/or Whole-body MRI and PET-CT scans of a subject. For example, in certain embodiments, the sample processing system 2490 may be or may include a Next-generation sequencer. In various embodiments, the EHR system 2490 may include, may be, or may employ, various computing devices that include health records of patients and study subjects (including devices of hospitals, clinics, healthcare practitioners, etc.), obtained from various sources, such as entries by healthcare practitioners, sample processing system 2480, university and hospital systems, government agency systems, etc.

In various embodiments, the computing device 2410 (or multiple computing devices) may be used to control, and receive signals acquired via, components of sample processing system 2480. The computing device 2410 may include one or more processors and one or more volatile and non-volatile memories for storing computing code and data that are captured, acquired, recorded, and/or generated. The computing device 2410 may include a control unit 2415 that in certain embodiments may be configured to exchange control signals with sample processing system 2480, allowing the computing device 2410 to be used to control, for example, processing of samples and/or scans and/or delivery of data generated and/or acquired through processing of samples and/or scans.

In various embodiments, computing device 2410 may include a data acquisition unit 2420 that may be configured to exchange control signals, or otherwise communicate, with sample processing system 2480 (or components thereof) and/or EHR system 2490, allowing the computing device 2410 to be used to control the capture of physiological data and/or signals via sensors of the sample processing system 2480, retrieve data or signals (e.g., from sample processing system 2480, EHR system 2490, and/or memory devices where data is stored), and direct transfer of data or signals (e.g., to sample processing system 2490 as feedback thereto, to EHR system 2490, to memory for storage, and/or to other systems or devices).

In various embodiment, a data analyzer 2425 may direct analysis of the data and signals, and output analysis results. Data analyzer 2425 may be used, for example, to transform raw data captured or obtained via sample processing system 2480 and/or EHR system 2490, and may employ pre-processing procedures involved in generating a training dataset. For example, in some implementations, data may be generated as a multi-dimensional array or vector with values representing, and to prevent the machine learning system from overemphasizing certain readings, values may be normalized to a predetermined range (e.g. 0-1, 0-100, or any other such range). The normalization may comprise linear rescaling, or may be a more complex function. In some implementations, dimension reduction may be performed to reduce large and sparse arrays or vectors. In some implementations, feature recognition may be performed to select a subset of features for further analysis, such as principal component analysis.

In various embodiments, a machine learning system 2430 may be used to implement various machine learning functionality discussed herein. Machine learning system 2430 may include a training engine 2435 configured to train predictive models using, for example, data obtained from or via data acquisition unit 2420 and/or processed data obtained from or via data analyzer 2425. The training engine 2435 may, for example, generate or obtain training datasets from or via data analyzer 2425 and may perform validation of datasets. The training engine 2435 may comprise a feature analyzer used to evaluate features by, for example, quantifying the impact of each feature on the developed model. Such a feature analyzer may, for example, uncover clinically important features that were globally predictive of the outcome, and may determine, for example, contributions of all features, or the top features (e.g., the top 2, top 5, top 10, top 15, top 20, top 25, top 30, etc.) on individual predictions. Features may be selected based on a threshold, such a percent contribution to predicting a medical condition, such as 0.5%, 1%, 2%, 5%, 10%, etc. A testing and application engine 2440 may be configured to test and apply models trained via training engine 2435 to, for example, study subject and/or patient data from data acquisition unit 2420 and/or data analyzer 2425.

In various embodiments, a transceiver 2445 allows the computing device 2410 to exchange readings, control commands, and/or other data with sample processing system 2480 (or components thereof) and/or EHR system 2490 (or components thereof). The transceiver 2445 may additionally or alternatively include a network interface permitting the computing device 2410 to communicate with other remote devices and systems via, for example, a telecommunications network such as the internet. One or more user interfaces 2450 allow the computing device 2410 to receive user inputs (e.g., via a keyboard, touchscreen, microphone, camera, etc.) and provide outputs (e.g., via a touchscreen or other display screen, audio speakers, haptic devices, etc.). A display screen may be employed, for example, to provide real time or near real time waveforms or other readings or measurements obtained via sensors being used to capture physiological data from subjects and patients. The computing device 2410 may additionally include one or more databases 2455 (stored in, e.g., one or more computer-readable non-volatile memory devices) for storing, for example, data and analyses obtained from or via data acquisition unit 2420, data analyzer 2425, machine learning system 2430 (e.g., training engine 2435 and/or testing and application engine 2440), sample processing system 2480, and/or EHR system 2490. In some implementations, database 2455 (or portions thereof) may alternatively or additionally be part of another computing device that is co-located or remote and in communication with computing device 2410, sample processing system 2480 (or components thereof), and/or EHR system 2490.

In one aspect, the present disclosure provides a method of training a machine learning classifier for estimating risk of cancer-associated venous thromboembolism (VTE) in cancer patients comprising: (a) receiving data on a cohort of subjects, the subjects in the cohort having a plurality of cancer types; (b) generating a training dataset based on the received data, wherein the training dataset comprises a plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and (c) applying a machine learning method to the training dataset to develop the machine learning classifier for estimating risk of cancer-associated VTE in cancer patients, wherein applying the machine learning method comprises: applying a machine learning technique to the training dataset; performing hyperparameter optimization to identify one or more machine learning models with an accuracy that exceeds an accuracy threshold for the classifier; and determining an optimal operating-point threshold based on optimization of sensitivity and specificity of the receiver operating characteristic (ROC) curves for the training dataset; wherein the classifier is configured to receive the plurality of features for cancer patients and generate predictors for risk of cancer-associated VTE in cancer patients. The subjects in the cohort may be chemotherapy-naïve or may have received systemic chemotherapy. Additionally or alternatively, in certain embodiments, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma. In any and all embodiments of the methods disclosed herein, the plurality of features does not comprise activated partial thromboplastin time, prothrombin time, or body mass index (BMI).

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

Additionally or alternatively, in some embodiments of the methods disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

In any of the preceding embodiments, the method further comprises applying the classifier to data on a cancer patient to generate a predictor, and determining whether the cancer patient is at risk for cancer-associated VTE based on the predictor and the operating-point threshold. In some embodiments, the predictor comprises a cumulative incidence function (CIF) for cancer-associated VTE.

In any of the foregoing embodiments, the method further comprises administering an effective amount of anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in some embodiments of the methods disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In one aspect, the present disclosure provides a method of estimating risk of cancer-associated venous thromboembolism (VTE) in a cancer patient using a machine learning classifier, the method comprising: receiving patient data corresponding to a plurality of features for the cancer patient; applying the machine learning classifier to the patient data to generate a predictor; and determining whether the cancer patient is at risk for cancer-associated VTE based on the predictor and an operating-point threshold, wherein the machine learning classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. In some embodiments, the method further comprises administering an effective amount of anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, and enoxaparin. Additionally or alternatively, in some embodiments, the predictor comprises a cumulative incidence function (CIF) for cancer-associated VTE. The subjects in the cohort may be chemotherapy-naïve or may have received systemic chemotherapy. In any of the preceding embodiments of the methods disclosed herein, the plurality of features for the cancer patient are determined by assaying blood and/or sequencing tumor DNA.

Additionally or alternatively, in certain embodiments, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma. In any and all embodiments of the methods disclosed herein, the plurality of features does not comprise activated partial thromboplastin time, prothrombin time, or body mass index (BMI).

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

Additionally or alternatively, in some embodiments of the methods disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

Additionally or alternatively, in some embodiments of the methods disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In any and all embodiments of the methods disclosed herein, the plurality of features for each subject in the cohort are determined by assaying blood and/or sequencing tumor DNA.

In any and all embodiments of the methods disclosed herein, the cancer-associated VTE is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

In another aspect, the present disclosure provides a machine learning system for training a machine learning classifier for estimating risk of cancer-associated venous thromboembolism (VTE) in cancer patients, the system comprising a processor and a memory with instructions which, when executed by the processor, cause the processor to: (a) receive data on a cohort of subjects, the subjects in the cohort having a plurality of cancer types; (b) generate a training dataset based on the received data, wherein the training dataset comprises a plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent, and wherein the plurality of features does not include neutrophil count and platelet count; and (c) apply a machine learning method to the training dataset to develop the machine learning classifier for estimating risk of cancer-associated VTE in cancer patients; wherein applying the machine learning method comprises: applying a machine learning technique to the training dataset; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. The subjects in the cohort may be chemotherapy-naïve or may have received systemic chemotherapy.

In any and all embodiments of the systems disclosed herein, the plurality of features does not comprise activated partial thromboplastin time, prothrombin time, or body mass index (BMI).

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

Additionally or alternatively, in some embodiments of the systems disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

Additionally or alternatively, in certain embodiments of the systems disclosed herein, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

In any of the preceding embodiments of the systems described herein, the instructions further cause the processor to apply the machine learning classifier to data on a cancer patient to generate a predictor, and determine whether the cancer patient is at risk for cancer-associated VTE based on the predictor and the operating-point threshold. In some embodiments, the predictor comprises a cumulative incidence function (CIF) for cancer-associated VTE.

In any of the foregoing embodiments of the systems described herein, the instructions further cause the processor to recommend an anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in some embodiments of the systems disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In yet another aspect, the present disclosure provides a computing system for estimating risk of cancer-associated venous thromboembolism (VTE) in a cancer patient, the computing system comprising a processor and a memory with instructions which, when executed by the processor, cause the processor to: receive patient data corresponding to a plurality of features for the cancer patient; apply a machine learning classifier to the patient data to generate a predictor; and determine whether the cancer patient is at risk for cancer-associated VTE based on the predictor and an operating-point threshold, wherein the classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

In any and all embodiments of the systems disclosed herein, the plurality of features does not comprise activated partial thromboplastin time, prothrombin time, or body mass index (BMI).

Additionally or alternatively, in some embodiments of the systems disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

In any of the preceding embodiments of the systems described herein, the instructions further cause the processor to recommend an anticoagulant therapy to the cancer patient predicted to be at risk for cancer-associated VTE based on the predictor and the operating-point threshold. In some embodiments, the predictor comprises a cumulative incidence function (CIF) for cancer-associated VTE. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in certain embodiments of the systems disclosed herein, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

Additionally or alternatively, in some embodiments of the systems disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In any and all embodiments of the systems disclosed herein, the plurality of features for each subject in the cohort are determined by assaying blood and/or sequencing tumor DNA.

In one aspect, the present disclosure provides a non-transitory computer-readable storage medium comprising instructions which, when executed by a processor of a machine learning system, configure the machine learning system to train a machine learning classifier to estimate risk of cancer-associated venous thromboembolism (VTE) in cancer patients, wherein the instructions are configured to cause the processor to: (a) receive data on a cohort of subjects, the subjects in the cohort having a plurality of cancer types; (b) generate a training dataset based on the received data, wherein the training dataset comprises a plurality of features for each subject in the cohort, the plurality of features comprising (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent, and wherein the plurality of features does not include neutrophil count and platelet count; and (c) apply a machine learning method to the training dataset to develop the machine learning classifier for estimating risk of cancer-associated VTE in cancer patients; wherein applying the machine learning method comprises: applying a machine learning technique to the training dataset; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients. The subjects in the cohort may be chemotherapy-naïve or may have received systemic chemotherapy.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

In any and all embodiments of the computer-readable storage medium disclosed herein, the plurality of features does not comprise activated partial thromboplastin time, prothrombin time, or body mass index (BMI).

Additionally or alternatively, in some embodiments of the computer-readable storage medium disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

In any of the preceding embodiments of the computer-readable storage medium described herein, the instructions further cause the processor to apply the machine learning classifier to data on a cancer patient to generate a predictor, and determine whether the cancer patient is at risk for cancer-associated VTE based on the predictor and the operating-point threshold. In some embodiments, the predictor comprises a cumulative incidence function (CIF) for cancer-associated VTE.

Additionally or alternatively, in certain embodiments of the computer-readable storage medium disclosed herein, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

In any of the preceding embodiments of the computer-readable storage medium described herein, the instructions further cause the processor to recommend an anticoagulant therapy to the cancer patient predicted to be at risk for cancer-associated VTE based on the predictor and the operating-point threshold. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in some embodiments of the computer-readable storage medium disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In another aspect, the present disclosure provides a non-transitory computer-readable storage medium comprising instructions which, when executed by a processor of a computing system, configure the computing system to estimate risk of cancer-associated venous thromboembolism (VTE) in a cancer patient, wherein the instructions are configured to cause the processor to: receive patient data corresponding to a plurality of features for the cancer patient; apply a machine learning classifier to the patient data to generate a predictor; and determine whether the cancer patient is at risk for cancer-associated VTE based on the predictor and an operating-point threshold, wherein the classifier is trained by: (a) receiving cohort data on a cohort of subjects, the subjects in the cohort having a plurality of cancer types; (b) generating a training dataset based on the received cohort data, wherein the training dataset comprises the plurality of features for each subject in the cohort, wherein the plurality of features comprises (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and (c) applying a machine learning method to the training dataset to develop the machine learning classifier for estimating risk of cancer-associated VTE, wherein applying the machine learning method comprises: applying a machine learning technique to the training dataset; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; wherein the machine learning classifier is configured to receive the plurality of features for cancer patients and generate predictors for risk of cancer-associated VTE in cancer patients.

The machine learning technique may model survival outcomes with competing risks. In some embodiments, the machine learning technique is a random forest technique, and the one or more machine learning models are random forest models. Additionally or alternatively, in certain embodiments, the machine learning classifier is an ensemble learning random forest classifier. In other embodiments, the machine learning technique is a deep neural network technique, and the one or more machine learning models are neural network models. Additionally or alternatively, in some embodiments, the machine learning classifier is a neural network classifier.

Additionally or alternatively, in some embodiments, performing the hyperparameter optimization comprises performing an exhaustive grid search technique. In other embodiments, performing the hyperparameter optimization comprises use of tree-structured Parzen estimators.

In any and all embodiments of the computer-readable storage medium disclosed herein, the plurality of features does not comprise activated partial thromboplastin time, prothrombin time, or body mass index (BMI).

Additionally or alternatively, in some embodiments of the computer-readable storage medium disclosed herein, the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. In other embodiments, the plurality of features further comprises time from cancer diagnosis, tumor mutation burden score and cancer somatic alterations in oncogenes or tumor suppressor genes. In certain embodiments, the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels. Additionally or alternatively, in some embodiments, the cancer somatic alterations in oncogenes or tumor suppressor genes comprise AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, and TP53. In certain embodiments, a lower weight is assigned to cancer somatic alterations in oncogenes or tumor suppressor genes.

In any of the preceding embodiments of the computer-readable storage medium described herein, the instructions further cause the processor to recommend an anticoagulant therapy to the cancer patient predicted to be at risk for cancer-associated VTE based on the predictor and the operating-point threshold. In some embodiments, the predictor comprises a cumulative incidence function (CIF) for cancer-associated VTE. Examples of anticoagulant therapy include, but are not limited to, apixaban, betrixaban, dabigatran, edoxaban, fondaparinux, heparin, rivaroxaban, warfarin, Xa inhibitors, and enoxaparin.

Additionally or alternatively, in certain embodiments of the computer-readable storage medium disclosed herein, the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

Additionally or alternatively, in some embodiments of the computer-readable storage medium disclosed herein, the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor. In some embodiments, the cancer patient is chemotherapy-naïve or has received/is receiving systemic chemotherapy.

In any of the preceding embodiments of the computer-readable storage medium disclosed herein, the plurality of features for the cancer patient are determined by assaying blood and/or sequencing tumor DNA.

### EXAMPLES

The present technology is further illustrated by the following Examples, which should not be construed as limiting in any way.

### Example 1: Experimental Methods

### Cohort

The cohort at large consisted of adult cancer patients who had MSK-IMPACT™ (Memorial Sloan Kettering Integrated Mutation Profiling of Actionable Cancer Targets) sequencing panel performed on their solid tumor malignancy between 2014 and 2019. All sequencing included a patient specific normal control.

VTE was defined as pulmonary embolism or lower extremity deep vein thrombosis (DVT). Lower extremity DVT included thrombi involving the common iliac vein, external iliac vein, common femoral vein, superficial femoral vein, deep femoral vein, popliteal vein, peroneal vein, anterior tibial vein, posterior tibial vein, or a deep calf vein. All such events were included regardless of the presence of symptoms. A VTE episode was considered cancer-associated if it occurred after or within the 365 days preceding a diagnosis of solid neoplasm. Events were detected using a review of medication prescriptions, keyword searches of radiology studies and the CEDARS NLP-based pipeline for patients who were included in the cohort between 2014 and 2016, as described elsewhere.⁴¹ Patients who had MSK-IMPACT™ performed between 2017 and 2019 were assessed only using CEDARS.⁴³ Parameters for this second CEDARS VTE event detection step are listed in Table 1.

TABLE 1

| Tokens used by CEDARS to detect sentences with a potential venous | |
|---|---|
| thromboembolic event*: | |
| Token | Endpoint |
| clot | thromboembolism |
| deep vein thrombosis | deep vein thrombosis |
| deep venous thrombosis | deep vein thrombosis |
| dvt | deep vein thrombosis |
| emboli | thromboembolism |
| embolism | thromboembolism |
| embolus | thromboembolism |
| pe | pulmonary embolism |
| phlebitis | thromboembolism |
| pulmonary emboli | pulmonary embolism |
| pulmonary embolism | pulmonary embolism |
| pulmonary embolus | pulmonary embolism |
| thrombi | thromboembolism |
| thromboembolic | thromboembolism |
| thromboembolism | thromboembolism |
| thrombophlebitis | venous thromboembolism |
| thrombose | thromboembolism |
| thrombosis | thromboembolism |
| thrombotic | thromboembolism |
| thrombus | thromboembolism |
| vte | venous thromboembolism |

*Sentences reviewed by an adjudicator before final classification; see cedars.io for detailed methods

All detected events were reviewed by two adjudicators, always including a hematologist. A random subset of patients was audited manually to estimate sensitivity and specificity of the automatic event detection algorithms (see infra).

Patients entered the cohort once their MSK-IMPACT™ results were reported in the clinical information system and were censored at the time of their last clinical note. These patients were included in the analysis regardless of timing for chemotherapy administration, as reporting a more generalizable model was considered desirable. Individuals were excluded if they had sustained an episode of cancer-associated thrombosis before the MSK-IMPACT™ result was reported.

### Random Audit Results

200 individuals were randomly selected in the cohort at large, for whom 28 cancer-associated VTE episodes had been previously recorded in the reference dataset. Hematology and anticoagulation clinic notes were examined for all 200 patients; no missed cancer-associated VTE episode was found. All 28 previously entered VTE episodes were examined for accuracy by reviewing pertinent clinical notes and radiology records. Two errors were found. In one case the data was correct as recorded initially based on information available, but additional notes entered later were uncovered during the audit which indicated timing was wrong and the VTE event was older than previously thought and not cancer-associated. In the second case, the cancer-associated VTE episode was found to have actually occurred 5 months before the date entered in the reference dataset.
Sensitivity: 100%
Specificity: 99%

### Choice of Machine Learning Algorithms

Several reliable machine learning (ML) classification algorithms exist; however, the choice was made early on to use VTE time-to-event as the main endpoint of interest. Without wishing to be bound by theory, it is believed that predicting the cumulative incidence function (CIF) of VTE would be more useful than a unique probability at 6 months, since it would allow the end user to effectively decide the time horizon they want to apply. Also, a set of event times contains more information than a limited assessment at an arbitrary cutoff, thus increasing the likelihood that the model is more accurate. Lastly, there was no computational cost to use a time-to-event endpoint in this use case, as opposed to a clinical prediction rule for which such an approach would be impractical.

One limitation of using a dataset spanning several years of observation is that a large number of individuals who may die before being censored. This is especially true for patients with cancer, even more so for the MSK-IMPACT™ cohort in which most participants have metastatic disease and a shortened life expectancy. Patient death is a competing risk since its occurrence will preclude the incidence of the primary event of interest (VTE). Conventional approaches, such as the semi-parametric standard Cox proportional hazard (CPH) model or the cause-specific hazard (CSH) model would not be appropriate for this use case because they do not account for the competing risk of death in the estimation process. CPH ignores competing events completely whereas the CSH model treats them as censoring. Counting competing events as censoring results in an upward biased estimation of risk for the primary event. In this regard, the bias associated with Kaplan-Meier estimates of VTE risk in patients with cancer and a high mortality rate has been discussed previously.⁴⁴ The remedy for this problem is to perform an adjustment for the competing risk of death. Several algorithms can be used for this purpose. The methods of the present technology used Fine-Gray (F&G) regression as a simple, basic baseline approach, along with Random Survival Forests (RSF) and deep learning for survival analysis as implemented by the DeepHit algorithm, based on the extent of published experience with these methods for competing risks.

Details on computing environment, statistical packages and main functions are provided below.

### Computing Environment, Statistical Packages and Main Functions:

R version 4.0.5, running on CentOS Linux 7 (Core)

Python version 3.8.13, running on Ubuntu 20.04.1 (LTS) GCC 7.5.0

### Fine and Gray (F&G) Model

F&G was implemented via the FGR function from the “riskRegression” package in R, which is a wrapper for the crr function from the R cmprsk package.

### Random Survival Forests (RSF)

RSF model was implemented using the R “randomForestSRC” package.

### Deep Survival Learning (DeepHit)

The DeepHit algorithm was implemented in Pytorch by modifying the model from Kvamme et al., Journal of Machine Learning Research. 2019; 20, including ensembles with bootstrap aggregation to reduce variance in the model predictions. Two corresponding distinct output layers were used in the model, corresponding to cancer-associated VTE and death.

### Imputation and Standardization

The IterativeImputer function implemented in Python was used for building an imputing model on the training dataset which was then used to impute both the training and the validation dataset. Continuous predictors used for the DeepHit model were standardized to zero mean and unit variance using the StandardScaler function in Python to ensure stable training of the deep model.

### Covariates Used in the Models

Predictors were grouped by elementary sets, the latter were combined to form the 11 final covariate sets.

### Elementary Sets:

BASIC=age, sex, cancer type (bladder, breast, colorectal, esophagogastric, gynecological, head and neck, hepatobiliary, high-grade glioma, low-grade glioma, lung, melanoma, pancreatic adenocarcinoma, prostate adenocarcinoma, renal, soft tissue sarcoma or other), sample type (local or metastatic), time from tumor sampling, time from cancer diagnosis

BASIC_MOD=age, sex, cancer type, sample type, time from tumor sampling

CHEMO=time elapsed since last systemic treatment (capped at 28 days), one value for each agent type (alkylating, antibiotic, antimetabolite, antimitotic, cyclin-dependent kinase inhibitor, epidermal growth factor receptor inhibitor, immune, multikinase inhibitor, PARP (poly ADP-ribose polymerase) inhibitor, platin, SERM (selective estrogen receptor modulator), VEGF (vascular endothelial growth factor) inhibitor or other); if systemic therapy was prescribed before cohort entry but not started yet, covariate value set to zero.

CHEMO_BINARIZED=chemotherapy administered in the last month or not

GENETIC=tumor mutation burden score, presence or absence of somatic genetic alteration for any of the genes with alteration frequency ≥1.5%. Gene list as follows: AKT1, APC, ARID1A, ARID1B, ATM, ATRX, BAP1, BCOR, BRAF, BRCA2, 'CCND1, CCNE1, CDH1, CDK4, CDKN2A, CDKN2B, CREBBP, CTCF, CTNNB1, EGFR, ERBB2, ERG, FAT1, FBXW7, FGF19, FGFR1, FOXA1, GATA3, IDH1, KDM6A, KIT, KRAS, MAP3K1, MCL1, MDM2, MLL2, MLL3, MYC, NF1, NRAS, PAK1, PBRM1, PIK3CA, PIK3R1, PTEN, RB1, RBM10, RNF43, SETD2, SMAD4, SMARCA4, SOX9, STK11, TERT, TP53

BASIC_LAB=albumin, hemoglobin

ADDITIONAL_LAB=sodium, potassium, chloride, calcium, CO₂, glucose, urea, creatinine, total protein, AST (aspartate transaminase), ALT (alanine transaminase), total bilirubin, alkaline phosphatase

### Final 11 Final Covariate Sets:

(1) EXTENSIVE_MOD=BASIC, CHEMO, GENETIC, BASIC_LAB
(2) EXTENSIVE=BASIC, CHEMO, GENETIC, BASIC_LAB, ADDITONAL_LAB
(3) NO_LABS=BASIC, CHEMO, GENETIC
(4) SIMPLE=BASIC, CHEMO
(5) LIMITED=BASIC, CHEMO, BASIC_LAB, ADDITONAL_LAB
(6) GENETIC_ONLY=BASIC, GENETIC
(7) GENETIC_CANCER_TYPE=cancer type, GENETIC
(8) CANCER_TYPE_ONLY=cancer type
(9) BASIC_GENETIC=BASIC, CHEMO_BINARIZED, GENETIC
(10) BASIC_CHEMO=BASIC_MOD, CHEMO, BASIC_LAB
(11) BASIC_BINARIZED=BASIC_MOD, CHEMO_BINARIZED

### Hyperparameter Tuning

For the RSF model, the optimal values for three hyperparameters (ntree, mtry and nodesize) were determined using an exhaustive grid search algorithm. The optimal combination maximized Antolini C-Index separately for each of the 11 covariate sets. Given the size of the training dataset, we decided to fix ntree to 500 and created a two-dimensional grid using 10 values (2 to 20 with step size of 2) of mtry and 40 values (2 to 80 with step size of 2) of nodesize. For the DeepHit model, the “hyperopt” package in Python was used to determine the optimal combination of the 10 hyperparameters. The search space for the hyperopt algorithm was defined by creating profiles for each of the 10 hyperparameters using distribution of their possible values. The objective function for the tree-structured Parzen optimization algorithm in the “hyperopt” package was defined to maximize the Antolini C-index.

The code and final models are available through the web page motrec.ai. The F&G approach^{45,46} extends the CPH model by directly estimating the effect of each covariate on the CIF. The RSF⁴⁶ model for competing risks is a modification of the non-competing RSF model from Ishwaran et al which is built using Breiman's Random Forest model framework. RSF is a highly adaptive non-linear model which does not rely on pre-specification of the higher-order interaction variables and is not restricted by the proportional hazards assumption. DeepHit, first described by Lee et al, allows the relationship between the covariates and risk estimate to change over time.^{47,48} To handle competing risks, this approach uses a common neural subnet and risk-specific subnets.

Model Training. Hyperparameter Tuning and Evaluation of Model Performance

The entire dataset was randomly partitioned into a training set comprising 80% of individuals and a validation set with the remainder of the cohort, stratifying by outcome (VTE or death). The subset of patients who could have their Khorana Score (KS) calculated was also evaluated separately (“KS subset”). In this group, the KS was assessed when an individual was prescribed systemic cancer treatment, if this occurred in the first 6 months after MSK-IMPACT™ report and there had been no treatment in the past year. The 6-month window restriction was applied to allow for a reliable comparison with models featuring genetic predictors. In the KS subset, predictions were made using laboratory and pharmacy data updated at the time the KS was derived; genomic data was the same as reported in the index IMPACT report.

Iterative imputation was used to handle missing data. Continuous predictors used for the DeepHit model were standardized to zero mean and unit variance. Predictors were selected based on prior knowledge and their actual contribution to predicting VTE events in the training set, using repeated 5-fold cross-validation on the training set. Available covariates included age, gender, cancer type, metastatic status, time from tumor sampling (i.e. biopsy or surgical resection of tissue sample), time from cancer diagnosis, time elapsed since last chemotherapy administered (stratified by pharmacological class), routine laboratory test results (hemoglobin, total protein, albumin, sodium, potassium, chloride, blood urea nitrogen, creatinine, carbon dioxide, glucose, calcium, aspartate transaminase, alanine transaminase, total bilirubin, alkaline phosphatase), tumor mutation burden score and cancer somatic alterations in oncogenes or tumor suppressor genes included in the first generation of the MSK-IMPACT™ panel. This MSK-IMPACT™ assay was described in detail elsewhere.⁴⁹ Only oncogenic or potentially oncogenic alterations were retained, including mutations, copy number alterations and fusions. Neutrophil count, platelet count, activated partial thromboplastin time and prothrombin time were not used because those values tend to change daily and secondarily to influence from chemotherapy (for blood cell counts) and anticoagulation (for clotting times). Without wishing to be bound by theory, it is believed that even though those predictors might seemingly improve accuracy, the final model would be less generalizable to other healthcare systems with different approaches to laboratory testing.

Optimal hyperparameters for RSF and DeepHit were determined using a grid search and tree-structured Parzen estimators respectively. For the F&G model, default hyperparameters were used. Metrics for all three models were derived using repeated 5-fold cross-validation, with 4 repeats within each cross-validation run, giving 20 values of the metric that were averaged to generate the overall metric. The confidence interval was estimated based on the standard error of the averaged metric. The main metric selected to evaluate models was the time-dependent concordance index as originally derived by Antolini et al.⁵⁰ This measurement quantifies the ability of the predictive model to discriminate among subjects with different event times along the CIF continuum. An index of 1 indicates perfect concordance between model predicted risk and actual survival, while a value of 0.5 means random concordance. The model considered as potentially the most useful in clinical practice was selected based on results from the training set. This main model was re-fitted on the whole training set and evaluated on the validation set. All other models were considered secondary.

The sensitivity and PPV of fitted models were assessed and compared those to values obtained when applying the KS, as derived from the results of a large review.⁸ KS thresholds used were 2 and 3. These values were used due to the abundance of information on the efficacy of VTE pharmacological prophylaxis in patients with a KS greater or equal to 2.⁵¹ In order to ensure the quality of performance report for the risk prediction models described herein, the Transparent Reporting of a multivariable prediction model for Individual Prognosis or Diagnosis (TRIPOD) tool was used (see check-list in Supplementary Information).⁵²

TRIPOD Checklist:

| | | Development | | |
|---|---|---|---|---|
| Section/topic | Item | or validation? | Checklist item Page | Comment |
| Title and abstract | | | | |
| Title | 1 | D; V | Identify the study as | complete |
| | | | developing and/or validating | |
| | | | a multivariable prediction | |
| | | | model, the target population, | |
| | | | and the outcome to be | |
| | | | predicted | |
| Abstract | 2 | D; V | Provide a summary of | complete |
| | | | objectives, study design, | |
| | | | setting, participants, sample | |
| | | | size, predictors, outcome, | |
| | | | statistical analysis, results, | |
| | | | and conclusions. | |
| Introduction | | | | |
| Background | 3a | D; V | Explain the medical context | complete |
| and objectives | | | (including whether | |
| | | | diagnostic or prognostic) and | |
| | | | rationale for developing or | |
| | | | validating the multivariable | |
| | | | prediction model, including | |
| | | | references to existing | |
| | | | models. | |
| | 3b | D; V | Specify the objectives, | complete |
| | | | including whether the study | |
| | | | describes the development or | |
| | | | validation of the model, or | |
| | | | both. | |
| Methods | | | | |
| Source of data | 4a | D; V | Describe the study design or | complete |
| | | | source of data (e.g., | |
| | | | randomized trial, cohort, or | |
| | | | registry data), separately for | |
| | | | the development and | |
| | | | validation data sets, if | |
| | | | applicable. | |
| | 4b | D; V | Specify the key study dates, | complete |
| | | | including start of accrual; | |
| | | | end of accrual; and, if | |
| | | | applicable, end of follow-up. | |
| Participants | 5a | D; V | Specify key elements of the | complete |
| | | | study setting (e.g., primary | |
| | | | care, secondary care, general | |
| | | | population) including | |
| | | | number and location of | |
| | | | centres. | |
| | 5b | D; V | Describe eligibility criteria | complete |
| | | | for participants. | |
| | 5c | D; V | Give details of treatments | complete |
| | | | received, if relevant. | |
| Outcome | 6a | D; V | Clearly define the outcome | complete |
| | | | that is predicted by the | |
| | | | prediction model, including | |
| | | | how and when assessed. | |
| | 6b | D; V | Report any actions to blind | complete |
| | | | assessment of the outcome | |
| | | | to be predicted. | |
| Predictors | 7a | D; V | Clearly define all predictors | complete |
| | | | used in developing the | |
| | | | multivariable prediction | |
| | | | model, including how and | |
| | | | when they were measured. | |
| | 7b | D; V | Report any actions to blind | complete |
| | | | assessment of predictors for | |
| | | | the outcome and other | |
| | | | predictors. | |
| Sample size | 8 | D; V | Explain how the study size | complete |
| | | | was arrived at. | |
| Missing data | 9 | D; V | Describe how missing data | complete |
| | | | were handled (e.g., | |
| | | | complete-case analysis, | |
| | | | single imputation, multiple | |
| | | | imputation) with details of | |
| | | | any imputation method. | |
| Statistical | 10a | D | Describe how predictors | complete |
| analysis | | | were handled in the | |
| methods | | | analyses. | |
| | 10b | D | Specify type of model, all | complete |
| | | | model-building procedures | |
| | | | (including any predictor | |
| | | | selection), and method for | |
| | | | internal validation. | |
| | 10c | V | For validation, describe how | complete |
| | | | the predictions were | |
| | | | calculated. | |
| | 10d | D; V | Specify all measures used to | complete |
| | | | assess model performance | |
| | | | and, if relevant, to compare | |
| | | | multiple models. | |
| | 10e | V | Describe any model | complete |
| | | | updating (e.g., recalibration) | |
| | | | arising from the validation, | |
| | | | if done. | |
| Risk groups | 11 | D; V | Provide details on how risk | complete |
| | | | groups were created, if done. | |
| Development | 12 | V | For validation, identify any | complete |
| vs. validation | | | differences from the | |
| | | | development data in setting, | |
| | | | eligibility criteria, outcome, | |
| | | | and predictors. | |
| Results | | | | |
| Participants | 13a | D; V | Describe the flow of | complete |
| | | | participants through the | |
| | | | study, including the number | |
| | | | of participants with and | |
| | | | without the outcome and, if | |
| | | | applicable, a summary of the | |
| | | | follow-up time. A diagram | |
| | | | may be helpful. | |
| | 13b | D; V | Describe the characteristics | complete |
| | | | of the participants (basic | |
| | | | demographics, clinical | |
| | | | features, available | |
| | | | predictors), including the | |
| | | | number of participants with | |
| | | | missing data for predictors | |
| | | | and outcome. | |
| | 13c | V | For validation, show a | complete |
| | | | comparison with the | |
| | | | development data of the | |
| | | | distribution of important | |
| | | | variables (demographics, | |
| | | | predictors and outcome). | |
| Model | 14a | D | Specify the number of | complete |
| development | | | participants and outcome | |
| | | | events in each analysis. | |
| | 14b | D | If done, report the | not performed, |
| | | | unadjusted association | re: not |
| | | | between each candidate | pertinent for |
| | | | predictor and outcome. | deep learning |
| Model | 15a | D | Present the full prediction | available upon |
| specification | | | model to allow predictions | request |
| | | | for individuals (i.e., all | |
| | | | regression coefficients, and | |
| | | | model intercept or baseline | |
| | | | survival at a given time | |
| | | | point). | |
| | 15b | D | Explain how to use the | complete |
| | | | prediction model. | |
| Model | 16 | D; V | Report performance | complete |
| performance | | | measures (with CIs) for the | |
| | | | prediction model. | |
| Model updating | 17 | V | If done, report the results | complete |
| | | | from any model updating | |
| | | | (i.e., model specification, | |
| | | | model performance). | |
| Discussion | | | | |
| Limitations | 18 | D; V | Discuss any limitations of | complete |
| | | | the study (such as | |
| | | | nonrepresentative sample, | |
| | | | few events per predictor, | |
| | | | missing data). | |
| Interpretation | 19a | V | For validation, discuss the | complete |
| | | | results with reference to | |
| | | | performance in the | |
| | | | development data, and any | |
| | | | other validation data. | |
| | 19b | D; V | Give an overall | complete |
| | | | interpretation of the results, | |
| | | | considering objectives, | |
| | | | limitations, results from | |
| | | | similar studies, and other | |
| | | | relevant evidence. | |
| Implications | 20 | D, V | Discuss the potential clinical | complete |
| | | | use of the model and | |
| | | | implications for future | |
| | | | research. | |
| Other information | | | | |
| Supplementary | 21 | D; V | Provide information about | complete |
| information | | | the availability of | |
| | | | supplementary resources, | |
| | | | such as study protocol, Web | |
| | | | calculator, and data sets. | |
| Funding | 22 | D; V | Give the source of funding | complete |
| | | | and the role of the funders | |
| | | | for the present study. | |

### Example 2: The Integrated Model of the Present Technology Accurately Predicts Cancer-Associated Venous Thromboembolism Across Multiple Cancer Types

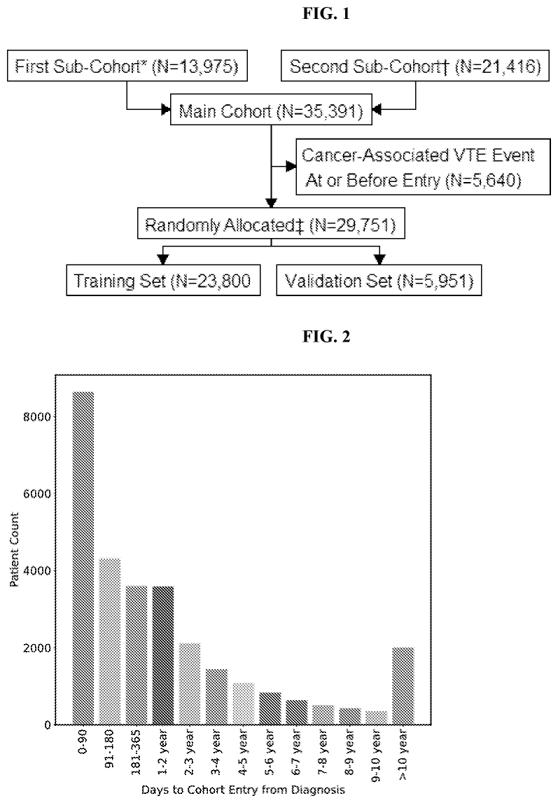

The aim of this study was to develop a machine learning algorithm to generate an accurate prediction of a patient's risk of developing cancer-associated venous thromboembolism by comprehensively integrating multiple clinical features associated with VTE, and to assess their predictive potential when combined in a single predictive framework. A total of 29,751 individuals were included in the final analysis. See FIG. 1 for flow diagram of the selection process. The characteristics of patients are shown in Table 2.

TABLE 2
Characteristics of Patients

| | Overall Cohort | | Training Set | | Validation set | |
|---|---|---|---|---|---|---|
| | (n = 29,751) | | (n = 23,800) | | (n = 5,951) | |
| Characteristic | Mean | SD | Mean | SD | Mean | SD |
| Age (years) | 60.8 | 13.9 | 60.8 | 13.9 | 60.5 | 13.9 |
| Tumor Burden Score | 8.1 | 20.0 | 8.1 | 20.2 | 7.9 | 19.4 |
| Hemoglobin (g/dL) | 12.0 | 1.9 | 12.0 | 1.9 | 12.0 | 1.8 |
| Platelet Count (10³/mcL) | 248.3 | 107.8 | 248.0 | 108.1 | 249.7 | 106.5 |
| Albumin (g/dL) | 4.0 | 0.5 | 4.0 | 0.5 | 4.0 | 0.5 |
| Time from Biopsy (days) | 321.3 | 678.1 | 323.5 | 687.4 | 312.4 | 639.6 |

| | N | % | N | % | N | % |
|---|---|---|---|---|---|---|
| Tumor type | | | | | | |
| Lung | 4,776 | 16 | 3,739 | 15 | 1,037 | 17 |
| Breast | 4,226 | 14 | 3,346 | 14 | 880 | 14 |
| Other | 3,974 | 13 | 3,166 | 13 | 808 | 13 |
| Colorectal | 3,103 | 10 | 2,527 | 10 | 576 | 9 |
| Gynecological | 2,926 | 9 | 2,336 | 9 | 590 | 9 |
| Prostate Adenocarcinoma | 1,974 | 6 | 1,572 | 6 | 402 | 6 |
| Pancreatic | 1,342 | 4 | 1,096 | 4 | 246 | 4 |
| Adenocarcinoma | | | | | | |
| Melanoma | 1,125 | 3 | 920 | 3 | 205 | 3 |
| Bladder | 1,033 | 3 | 845 | 3 | 188 | 3 |
| Soft Tissue Sarcoma | 1,053 | 3 | 845 | 3 | 208 | 3 |
| Esophagogastric | 947 | 3 | 778 | 3 | 169 | 2 |
| High-Grade Glioma | 913 | 3 | 743 | 3 | 170 | 2 |
| Hepatobiliary | 857 | 2 | 704 | 2 | 153 | 2 |
| Head and Neck | 643 | 2 | 513 | 2 | 130 | 2 |
| Renal | 590 | 1 | 463 | 1 | 127 | 2 |
| Low-Grade Glioma | 269 | 0 | 207 | 0 | 62 | 1 |
| Sample Type | | | | | | |
| Local Tumor | 18,526 | 62 | 14,807 | 62 | 3,719 | 62 |
| Metastasis | 11,225 | 37 | 8,993 | 37 | 2,232 | 37 |

SD: Standard Deviation

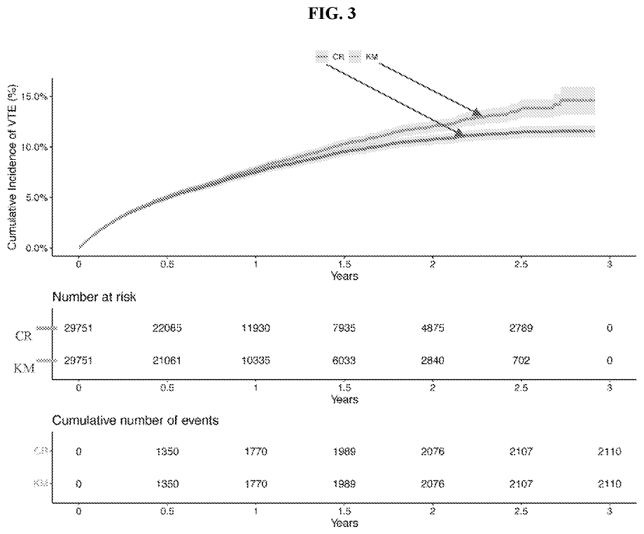

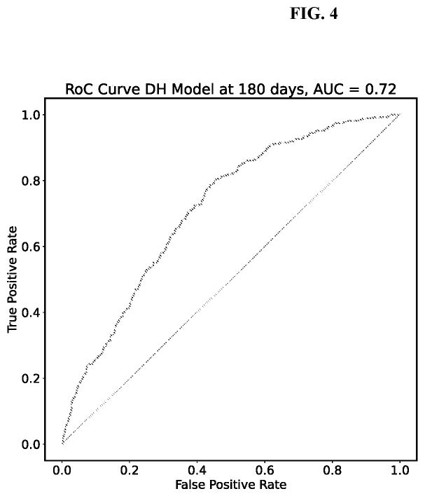

The median age was 60.8 years. The most frequent tumor type was lung, representing 16% of patients. Over half of the samples were from a metastatic site, with 62% of cases falling into this category. The median time from cancer diagnosis upon cohort entry was 256 days (IQR=79-1075 days, FIG. 2). The median observation time was 239 days. Cancer-associated VTE occurred during the first 6 months of observation in 1,338 (4.5%) of the patients. Cumulative incidence functions for this outcome were derived using Kaplan-Meier (KM) and competing risk (CR) estimators (FIG. 3). The 6-month cumulative VTE estimates using the KM method and the CR estimates are almost identical (5.0% vs. 5.1%); however, this almost non-existent bias gets more pronounced over time in the full observation cohort (15% vs. 12%). There are slightly fewer patients-at-risk in the KM curve compared with the CR curve (21,061 and 22,085 respectively) for the 6-month period and this difference is more pronounced when considering the full observation period (702 vs 2,789).

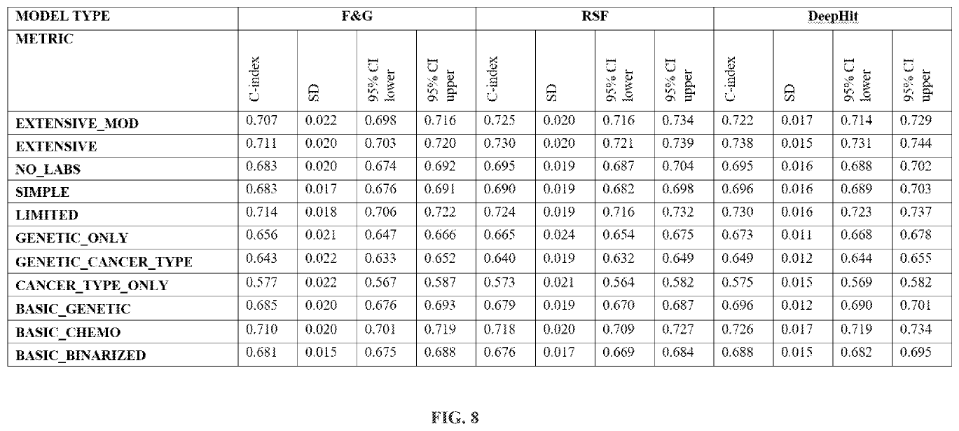

Models were derived using 11 covariate sets (see Methods). The three approaches (F&G, RSF and DeepHit) were applied to each covariate set on the training set. The Antolini C-index results for the training set (n=23,800) are provided in FIG. 8. The highest value was noted for the DeepHit model using the “extensive” covariate set, including demographics, cancer-specific characteristics, laboratory values, systemic treatment types and genomic predictors (C-index=0.738, 95% CI=0.731-0.744). This result was not significantly different from the one obtained with a RSF model (C-index=0.730, 95% CI=0.721-0.739), but was significantly higher than what was obtained using F&G regression (C-index=0.711, 95% CI=0.703-0.720). The DeepHit model using the same predictors but excluding genomic information (“limited” set) performed similarly (C-index=0.730, 95% CI=0.723-0.737). Given the absence of a significant improvement in concordance using genomic predictors on the full training set, the “limited” covariate set was selected. The DeepHit approach was retained, considering that increased complexity was justified by the potential to use transfer learning in the future.

Using the optimal hyperparameters derived from cross-validation, all models were re-fitted on the entirety of the training set and final metrics computed on the validation set (n=5,951). Confidence intervals were estimated with bootstrapping. Results using DeepHit for pertinent predictor sets are shown in Table 3.

TABLE 3
Performance of DeepHit Models on the Validation Set

| | | | Sensitivity, High Risk |
|---|---|---|---|
| | C-index | Sensitivity, Intermediate | Group‡ |
| Covariate Set | (95% CI) | Risk Group† (95% CI) | (95% CI) |
| Extensive | 0.722 (0.720-0.724) | 0.572 (0.555-0.589) | 0.285 (0.272-0.298) |
| Limited | 0.721 (0.719-0.723) | 0.638 (0.624-0.651) | 0.273 (0.259-0.286) |
| Cancer type with | 0.594 (0.592-0.597) | 0.070 (0.063-0.077) | 0.037 (0.032-0.041) |
| genetic predictors | | | |
| Cancer type alone | 0.557 (0.555-0.560) | 0.025 (0.016-0.034) | 0.003 (0.002-0.005) |

†Corresponding to a PPV of 8.9%, based on an estimate for a KS of 2 or more as reported in a meta-analysis from Mulder et al.⁸
‡Corresponding to a PPV of 11.0%, based on an estimate for a KS of 3 or more as reported in a meta-analysis from Mulder et al.⁸

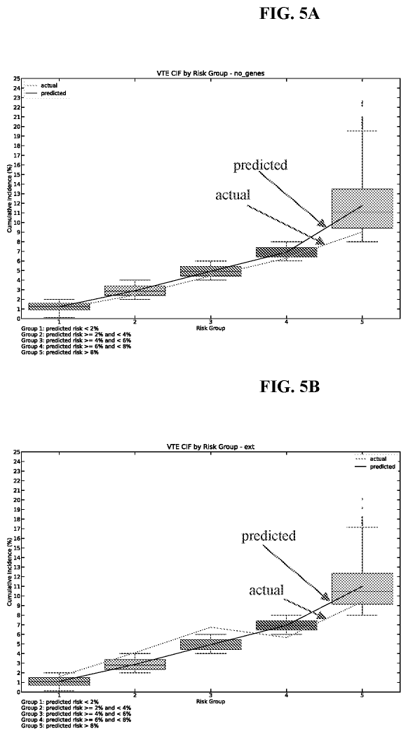

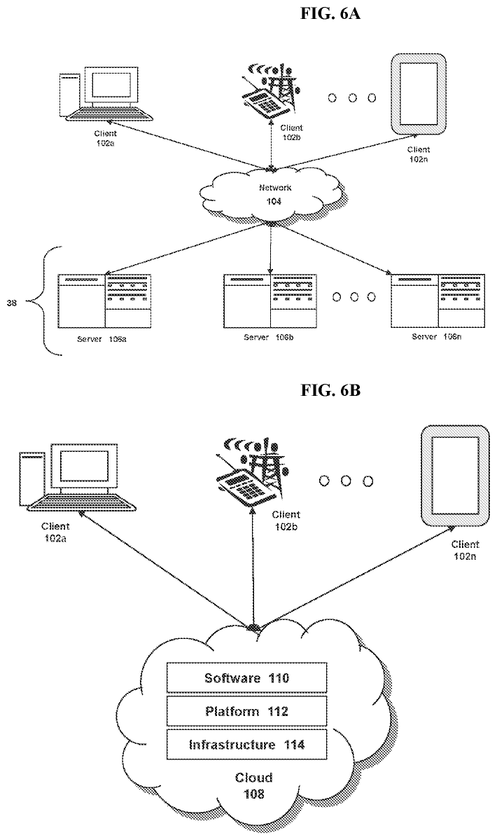

Using DeepHit and the “limited” covariate set, the Antolini C-index was 0.721 (95% CI=0.719-0.723) on the full validation set; see FIG. 4 for the responder operating characteristic (ROC) curve and FIGS. 5A-5B for the calibration plots. Sensitivity was 63.8% (95% CI=62.4%-65.1%) when PPV was fixed at 8.9% and 27.3% (95% CI=25.9%-28.6%) when PPV was fixed at 11.0%. Concordance was not significantly different with F&G (C-index=0.723, 95% CI=0.721-0.725) and RSF (C-index=0.720, 95% CI=0.718-0.722). Adding genomic predictors to a simple model based on cancer type alone significantly improved the main metric using DeepHit (C-index=0.594, 95% CI=0.592-0.597 vs C-index=0.557, 95% CI=0.555-0.560).

Concordance for the selected model (limited set of covariates) was preserved in the group of patients newly started on systemic therapy (“KS validation subset”, n=486), with an area under the receiver-operator characteristic curve (AUC ROC) at the 6-month mark of 0.751 for the selected DeepHit model. The AUC ROC using the KS was 0.587 for the KS validation subset and 0.621 for the whole KS subset. Metrics for the KS validation subset using the DeepHit model and the KS are shown in Table 4.

TABLE 4
Comparison Between the Khorana Score and the Selected DeepHit Model

| | | Sensitivity, Intermediate | Sensitivity, High |
|---|---|---|---|
| Model | C-index* | Risk Group† | Risk Group‡ |
| Khorana Score | 0.587 | 0.308 | 0.128 |
| Selected Model | 0.751 | 0.975 | 0.881 |

Results computed on the KS subset of the validation set (n = 486). PPV was 9.4% for a KS of 2 or more and 13.2% for a KS of 3 or more. Confidence intervals not computed due to small number of events (n = 5 for KS ≥3).
*C-index calculated as the area under the ROC curve at 6 months.
†Corresponding to a KS of 2 or more, or a fixed PPV of 9.4% for the selected model.
‡Corresponding to a KS of 3 or more, or a fixed PPV of 13.2% for the selected model.

PPV was 9.4% for a KS≥2 and 13.2% for a KS≥3. Sensitivity was 30.8% and 12.8% using a KS≥2 and 3 respectively, compared to 97.5% and 88.1% for the DeepHit model and using a fixed PPV of 9.4% and 13.2% respectively. Most patients in the KS subset were at low risk of VTE, as 74% of patients had a KS of 0 or 1 and only 26% had a KS≥2. This is compared to values of 53% and 47% respectively for a large meta-analysis of studies evaluating the KS.⁸ In this latter report, sensitivity was 55.2% (95% CI not provided) for a KS≥2 and 23.4% (95% CI=18.4%-29.4%) for a KS≥3.

Given the superior results for the DeepHit model with an extensive set of covariates in the KS subset, an additional analysis was conducted on the subset of all patients who had received systemic therapy in the prior 28 days or were going to start chemotherapy (n=3,306, within the validation set only). The model featuring the “extensive” set of covariates had the best performance and was found to be significantly better than the one without genetic predictors (i.e. “limited” set), with C-indexes of 0.678 (95% CI=0.676-0.681) and 0.660 (95% CI=0.658-0.662) respectively. Lastly, another subset analysis was performed for individuals who started observation less than one year after their initial cancer diagnosis. In this group of 3,321 patients from the validation set, the C-index was 0.743 (95% CI=0.741-0.746) for the DeepHit model featuring a “limited” set of predictors, compared to 0.733 (95% CI=0.731-0.736) for the model including genomic predictors.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Timp, J. F., Braekkan, S. K., Versteeg, H. H. & Cannegieter, S. C. Epidemiology of cancer-associated venous thrombosis. Blood 122, 1712-1723 (2013).
2. Falanga, A., Schieppati, F. & Russo, L. Pathophysiology 1. Mechanisms of Thrombosis in Cancer Patients. Cancer Treat Res 179, 11-36 (2019).
3. Horsted, F., West, J. & Grainge, M. J. Risk of venous thromboembolism in patients with cancer: a systematic review and meta-analysis. PLoS Med 9, e1001275 (2012).
4. Khorana, A. A., Francis, C. W., Culakova, E., Kuderer, N. M. & Lyman, G. H. Thromboembolism is a leading cause of death in cancer patients receiving outpatient chemotherapy. J Thromb Haemost 5, 632-634 (2007).
5. Khorana, A. A., et al. Rivaroxaban for Thromboprophylaxis in High-Risk Ambulatory Patients with Cancer. N Engl J Med 380, 720-728 (2019).
6. Carrier, M., et al. Apixaban to Prevent Venous Thromboembolism in Patients with Cancer. N Engl J Med 380, 711-719 (2019).
7. Khorana, A. A., Kuderer, N. M., Culakova, E., Lyman, G. H. & Francis, C. W. Development and validation of a predictive model for chemotherapy-associated thrombosis. Blood 111, 4902-4907 (2008).
8. Mulder, F. I., et al. The Khorana score for prediction of venous thromboembolism in cancer patients: a systematic review and meta-analysis. Haematologica 104, 1277-1287 (2019).
9. Ay, C., et al. Prediction of venous thromboembolism in cancer patients. Blood 116, 5377-5382 (2010).
10. Verso, M., Agnelli, G., Barni, S., Gasparini, G. & LaBianca, R. A modified Khorana risk assessment score for venous thromboembolism in cancer patients receiving chemotherapy: the Protecht score. Intern Emerg Med 7, 291-292 (2012).
11. Pelzer, U., Sinn, M., Stieler, J. & Riess, H. [Primary pharmacological prevention of thromboembolic events in ambulatory patients with advanced pancreatic cancer treated with chemotherapy?]. Dtsch Med Wochenschr 138, 2084-2088 (2013).
12. Cella, C. A., et al. Preventing Venous Thromboembolism in Ambulatory Cancer Patients: The ONKOTEV Study. Oncologist 22, 601-608 (2017).
13. Gerotziafas, G. T., et al. A Predictive Score for Thrombosis Associated with Breast, Colorectal, Lung, or Ovarian Cancer: The Prospective COMPASS-Cancer-Associated Thrombosis Study. Oncologist 22, 1222-1231 (2017).
14. Munoz Martin, A. J., et al. Multivariable clinical-genetic risk model for predicting venous thromboembolic events in patients with cancer. Br J Cancer 118, 1056-1061 (2018).
15. May, M. Eight ways machine learning is assisting medicine. Nat Med 27, 2-3 (2021).
16. Chamberlain, J. M., Chamberlain, D. B. & Zorc, J. J. Machine Learning and Clinical Prediction Rules: A Perfect Match? Pediatrics 146(2020).
17. Ferroni, P., et al. Validation of a Machine Learning Approach for Venous Thromboembolism Risk Prediction in Oncology. Dis Markers 2017, U.S. Pat. No. 8,781,379 (2017).
18. Jin, S., et al. Machine learning predicts cancer-associated deep vein thrombosis using clinically available variables. Int J Med Inform 161, 104733 (2022).
19. Lei, H., et al. Development and Validation of a Risk Prediction Model for Venous Thromboembolism in Lung Cancer Patients Using Machine Learning. Front Cardiovasc Med 9, 845210 (2022).
20. Carobbio, A., et al. Risk factors for arterial and venous thrombosis in WHO-defined essential thrombocythemia: an international study of 891 patients. Blood 117, 5857-5859 (2011).
21. Corrales-Rodriguez, L., et al. Mutations in NSCLC and their link with lung cancer-associated thrombosis: a case-control study. Thromb Res 133, 48-51 (2014).
22. Lee, Y. G., et al. Risk factors and prognostic impact of venous thromboembolism in Asian patients with non-small cell lung cancer. Thromb Haemost 111, 1112-1120 (2014).
23. Rumi, E., et al. Clinical effect of driver mutations of JAK2, CALR, or MPL in primary myelofibrosis. Blood 124, 1062-1069 (2014).
24. Ades, S., et al. Tumor oncogene (KRAS) status and risk of venous thrombosis in patients with metastatic colorectal cancer. J Thromb Haemost 13, 998-1003 (2015).
25. Qin, Y., Wang, X., Zhao, C., Wang, C. & Yang, Y. The impact of JAK2V617F mutation on different types of thrombosis risk in patients with essential thrombocythemia: a meta-analysis. Int J Hematol 102, 170-180 (2015).
26. Verso, M., et al. Incidence of Ct scan-detected pulmonary embolism in patients with oncogene-addicted, advanced lung adenocarcinoma. Thromb Res 136, 924-927 (2015).
27. Unruh, D., et al. Mutant IDH1 and thrombosis in gliomas. Acta Neuropathol 132, 917-930 (2016).
28. Davidsson, E., et al. Mutational status predicts the risk of thromboembolic events in lung adenocarcinoma. Multidisciplinary Respiratory Medicine 12(2017).
29. Zer, A., et al. ALK-Rearranged Non-Small-Cell Lung Cancer Is Associated With a High Rate of Venous Thromboembolism. Clin Lung Cancer 18, 156-161 (2017).
30. Dou, F., et al. Association between oncogenic status and risk of venous thromboembolism in patients with non-small cell lung cancer. Respir Res 19, 88 (2018).
31. Mir Seyed Nazari, P., et al. Combination of isocitrate dehydrogenase 1 (IDH1) mutation and podoplanin expression in brain tumors identifies patients at high or low risk of venous thromboembolism. J Thromb Haemost 16, 1121-1127 (2018).
32. Zugazagoitia, J., et al. Incidence, predictors and prognostic significance of thromboembolic disease in patients with advanced ALK-rearranged non-small cell lung cancer. Eur Respir J 51(2018).
33. Gervaso, L. P. S., et al. Molecular Subtyping to Predict Risk of Venous Thromboembolism in Patients with Advanced Lung Adenocarcinoma: A Cohort Study. Blood 134(2019).
34. Ng, T. L., et al. ROS1 Gene Rearrangements Are Associated With an Elevated Risk of Peridiagnosis Thromboembolic Events. J Thorac Oncol 14, 596-605 (2019).
35. Wang, J., et al. The EGFR-rearranged adenocarcinoma is associated with a high rate of venous thromboembolism. Ann Transl Med 7, 724 (2019).
36. Al-Samkari, H., et al. Impact of ALK Rearrangement on Venous and Arterial Thrombotic Risk in NSCLC. J Thorac Oncol 15, 1497-1506 (2020).
37. Alexander, M., et al. A multicenter study of thromboembolic events among patients diagnosed with ROS1-rearranged non-small cell lung cancer. Lung Cancer 142, 34-40 (2020).
38. Chiari, R., et al. ROS1-rearranged Non-small-cell Lung Cancer is Associated With a High Rate of Venous Thromboembolism: Analysis From a Phase II, Prospective, Multicenter, Two-arms Trial (METROS). Clin Lung Cancer 21, 15-20 (2020).
39. Dou, F., et al. Association of ALK rearrangement and risk of venous thromboembolism in patients with non-small cell lung cancer: A prospective cohort study. Thromb Res 186, 36-41 (2020).
40. Munoz-Unceta, N., et al. High risk of thrombosis in patients with advanced lung cancer harboring rearrangements in ROS1. Eur J Cancer 141, 193-198 (2020).
41. Dunbar, A., et al. Genomic profiling identifies somatic mutations predicting thromboembolic risk in patients with solid tumors. Blood 137, 2103-2113 (2021).
42. Roopkumar, J., et al. Risk of thromboembolism in patients with ALK- and EGFR-mutant lung cancer: A cohort study. J Thromb Haemost 19, 822-829 (2021).
43. Mantha, S. CEDARS—Clinical Event Detection and Recording System (cedars.io).
44. Ay, C., Posch, F., Kaider, A., Zielinski, C. & Pabinger, I. Estimating risk of venous thromboembolism in patients with cancer in the presence of competing mortality. J Thromb Haemost 13, 390-397 (2015).
45. Stensrud, M. J. & Hernan, M. A. Why Test for Proportional Hazards? JAMA 323, 1401-1402 (2020).
46. Schemper, M., Wakounig, S. & Heinze, G. The estimation of average hazard ratios by weighted Cox regression. Stat Med 28, 2473-2489 (2009).
47. Lee, C., Zame, W. R., Yoon, J. & van der Schaar, M. DeepHit: A Deep Learning Approach to Survival Analysis with Competing Risks. in The Thirty-Second AAAI Conference on Artificial Intelligence (AAAI-18) (Association for the Advancement of Artificial Intelligence, New Orleans, Louisiana, 2018).
48. Kvamme, H., Borgan, O. & Scheel, I. Time-to-Event Prediction with Neural Networks and Cox Regression. J Mach Learn Res 20(2019).
49. Cheng, D. T., et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. J Mol Diagn 17, 251-264 (2015).
50. Antolini, L., Boracchi, P. & Biganzoli, E. A time-dependent discrimination index for survival data. Stat Med 24, 3927-3944 (2005).
51. Bosch, F. T. M., et al. Primary thromboprophylaxis in ambulatory cancer patients with a high Khorana score: a systematic review and meta-analysis. Blood Adv 4, 5215-5225 (2020).
52. Collins, G. S., Reitsma, J. B., Altman, D. G. & Moons, K. G. Transparent reporting of a multivariable prediction model for individual prognosis or diagnosis (TRIPOD): the TRIPOD Statement. BMC Med 13, 1 (2015).
53. Tan, C., et al. A Survey on Deep Transfer Learning. in Artificial Neural Networks and Machine Learning—ICANN 2018 (eds. Kurková, V., Manolopoulos, Y., Hammer, B., Iliadis, L. & Maglogiannis, I.) 270-279 (Springer International Publishing, Cham, 2018).
54. Sanfilippo, K. M., et al. Standardization of risk prediction model reporting in cancer-associated thrombosis: Communication from the ISTH SSC subcommittee on hemostasis and malignancy. J Thromb Haemost (2022).

1. A method of training a machine learning classifier for estimating risk of cancer-associated venous thromboembolism (VTE) in cancer patients, comprising:
receiving data on a cohort of subjects, the subjects in the cohort having a plurality of cancer types;
generating a training dataset based on the received data, the training dataset comprising a plurality of features for each subject in the cohort, the plurality of features comprising (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and
applying a machine learning method to the training dataset to develop the machine learning classifier for estimating risk of cancer-associated VTE in cancer patients,
wherein applying the machine learning method comprises:
applying a machine learning technique to the training dataset;
performing hyperparameter optimization to identify one or more machine learning models with an accuracy that exceeds an accuracy threshold for the classifier; and
determining an optimal operating-point threshold based on optimization of sensitivity and specificity of the receiver operating characteristic (ROC) curves for the training dataset;
wherein the classifier is configured to receive the plurality of features for cancer patients and generate predictors for risk of cancer-associated VTE in cancer patients.

2. The method of claim 1, wherein the plurality of features does not comprise activated partial thromboplastin time, prothrombin time, or body mass index (BMI); or
wherein the plurality of features further comprises time from cancer diagnosis, tumor mutation burden score and cancer somatic alterations in oncogenes or tumor suppressor genes; or
wherein the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels; or
wherein the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase level.

3. (canceled)

4. (canceled)

5. (canceled)

6. The method of claim 1, wherein the machine learning technique is a random forest technique, and wherein the one or more machine learning models are random forest models; or
wherein the machine learning classifier is an ensemble learning random forest classifier; or
wherein the machine learning technique is a deep neural network technique, and wherein the one or more machine learning models are neural network models; or
wherein the machine learning classifier is a neural network classifier; or
wherein the machine learning technique models survival outcomes with competing risks.

7. (canceled)

8. (canceled)

9. (canceled)

10. (canceled)

11. The method of claim 1, wherein performing the hyperparameter optimization comprises performing an exhaustive grid search technique; or
wherein performing the hyperparameter optimization comprises use of tree-structured Parzen estimators.

12. (canceled)

13. The method of claim 1, further comprising applying the classifier to data on a cancer patient to generate a predictor, and determining whether the cancer patient is at risk for cancer-associated VTE based on the predictor and the operating-point threshold.

14. The method of claim 13, wherein the predictor comprises a cumulative incidence function (CIF) for cancer-associated VTE; or
wherein the cancer patient is chemotherapy-naïve or has received/is receiving systemic chemotherapy; or
wherein the method further comprises administering an effective amount of anticoagulant therapy to the cancer patient predicted to be at risk for cancer-associated VTE based on the predictor and the operating-point threshold, optionally wherein the anticoagulant therapy comprises one or more of apixaban, betrixaban, dabigatran, edoxaban, fondaparinux, heparin, rivaroxaban, warfarin, Xa inhibitors, or enoxaparin.

15. (canceled)

16. (canceled)

17. The method of claim 1, wherein the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma; or
wherein the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor; or
wherein the subjects in the cohort are chemotherapy-naïve or have received systemic chemotherapy.

18. (canceled)

19. (canceled)

20. (canceled)

21. A method of estimating risk of cancer-associated venous thromboembolism (VTE) in a cancer patient using a machine learning classifier, the method comprising:
receiving patient data corresponding to a plurality of features for the cancer patient;
applying the machine learning classifier to the patient data to generate a predictor; and
determining whether the cancer patient is at risk for cancer-associated VTE based on the predictor and an operating-point threshold, wherein the machine learning classifier is trained by:
receiving cohort data on a cohort of subjects, the subjects in the cohort having a plurality of cancer types;
generating a training dataset based on the received cohort data, the training dataset comprising the plurality of features for each subject in the cohort, the plurality of features comprising (i) age, (ii) sex, (iii) cancer type, (iv) status of tumor metastasis, (v) time from tumor sampling, (vi) blood albumin level, (vii) blood hemoglobin level, and (viii) time elapsed since last systemic treatment with at least one anti-cancer therapeutic agent and wherein the plurality of features does not include neutrophil count and platelet count; and
applying a machine learning method to the training dataset to develop the machine learning classifier for estimating risk of cancer-associated VTE,
wherein applying the machine learning method comprises:
applying a machine learning technique to the training dataset;
performing hyperparameter optimization to identify one or more machine learning models with an accuracy that exceeds an accuracy threshold for the machine learning classifier; and
determining the optimal operating-point threshold based on optimization of sensitivity and specificity of the receiver operating characteristic (ROC) curves for the training dataset;
wherein the machine learning classifier is configured to receive the plurality of features for cancer patients and generate predictors for risk of cancer-associated VTE in cancer patients.

22. The method of claim 21, further comprising administering an effective amount of anticoagulant therapy to the cancer patient predicted to be at risk for cancer-associated VTE based on the predictor and the operating-point threshold, optionally wherein the predictor comprises a cumulative incidence function (CIF) for cancer-associated VTE.

23. (canceled)

24. The method of claim 21, wherein the machine learning technique is a random forest technique, and wherein the one or more machine learning models are random forest models; or
wherein the machine learning classifier is an ensemble learning random forest classifier; or
wherein the machine learning technique is a deep neural network technique, and wherein the one or more machine learning models are neural network models; or
wherein the machine learning classifier is a neural network classifier; or
wherein the machine learning technique models survival outcomes with competing risks.

25. (canceled)

26. (canceled)

27. (canceled)

28. (canceled)

29. The method of claim 21, wherein performing the hyperparameter optimization comprises performing an exhaustive grid search technique; or
wherein performing the hyperparameter optimization comprises use of tree-structured Parzen estimators.

30. (canceled)

31. The method of claim 21, wherein the plurality of features does not comprise activated partial thromboplastin time, prothrombin time, or body mass index (BMI).

32. The method of claim 21, wherein the plurality of features further comprises time from cancer diagnosis, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels; or
wherein the plurality of features further comprises time from cancer diagnosis, tumor mutation burden score and cancer somatic alterations in oncogenes or tumor suppressor genes.

33. (canceled)

34. The method of claim 21, wherein the plurality of features further comprises time from cancer diagnosis, tumor mutation burden score, cancer somatic alterations in oncogenes or tumor suppressor genes, blood total protein levels, blood sodium levels, blood potassium levels, blood chloride levels, blood calcium levels, blood CO₂ levels, blood glucose levels, blood urea levels, blood creatinine levels, blood AST (aspartate transaminase) levels, blood ALT (alanine transaminase) levels, blood total bilirubin levels, and blood alkaline phosphatase levels.

35. The method of claim 22, wherein the anticoagulant therapy comprises one or more of apixaban, betrixaban, dabigatran, edoxaban, fondaparinux, heparin, rivaroxaban, warfarin, Xa inhibitors, or enoxaparin.

36. The method of claim 21, wherein the plurality of cancer types are selected from the group consisting of bladder cancer, breast cancer, colorectal cancer, esophagogastric cancer, gynecological cancer (e.g., uterine cancer, cervical cancer, ovarian cancer), head and neck cancer, hepatobiliary cancer, high-grade glioma, low-grade glioma, lung cancer, melanoma, pancreatic cancer, prostate cancer, renal cancer, and soft tissue sarcoma.

37. The method of claim 21, wherein the at least one anti-cancer therapeutic agent comprises one or more of an alkylating agent, an antibiotic, an antimetabolite, an antimitotic, a cyclin-dependent kinase inhibitor, an epidermal growth factor receptor inhibitor, a multikinase inhibitor, a PARP inhibitor, a platinum-based agent, a selective estrogen receptor modulator (SERM), and a VEGF inhibitor.

38. The method of claim 21, wherein the plurality of features for the cancer patient are determined by assaying blood and/or sequencing tumor DNA.

39. The method of claim 1, wherein the plurality of features for each subject in the cohort are determined by assaying blood and/or sequencing tumor DNA.

40. The method of claim 1, wherein the cancer-associated VTE is pulmonary embolism or lower extremity deep vein thrombosis (DVT), optionally wherein lower extremity DVT includes thrombi involving a common iliac vein, an external iliac vein, a common femoral vein, a superficial femoral vein, a deep femoral vein, a popliteal vein, a peroneal vein, an anterior tibial vein, a posterior tibial vein, or a deep calf vein.

41.-116. (canceled)
